(19) United States
(12) Patent Application Publication
(10) Pub. No.: US 2014/0309534 A1
(43) Pub. Date: Oct. 16, 2014

(54) PRESSURE PLATE DEVICE FOR ORTHOTIC SELECTION

(71) Applicant: ORTHERA INC., San Diego, CA (US)

(72) Inventors: Thomas Pichler, San Diego, CA (US); Nolan Bedford, San Diego, CA (US); Cristobal Gonzalez, San Diego, CA (US); Sonja Pichler, San Diego, CA (US); Craig Payea, San Diego, CA (US)

(73) Assignee: ORTHERA INC., San Diego, CA (US)

(21) Appl. No.: 14/251,483

(22) Filed: Apr. 11, 2014

Related U.S. Application Data
(60) Provisional application No. 61/811,590, filed on Apr. 12, 2013.

(51) Int. Cl. A61B 5/103

(52) U.S. Cl. CPC A61B 5/1036
USPC 600/476

(57) ABSTRACT

A foot pressure measurement and foot length measurement device employs a plurality of pressure sensors and an optical foot length measurement system. The device also includes a computer that can perform an analysis of the foot pressure and length data to select an appropriate orthotic device. The foot measurement device may be integrated into a kiosk. Orthotic devices that have myriad configurations are also disclosed.

### CROSS-REFERENCE TO RELATED APPLICATION(S)

This application claims priority to Provisional Application No. 61/811,590, filed Apr. 12, 2013, titled “PRESSURE PLATE DEVICE FOR ORTHOTIC SELECTION”, which is hereby incorporated by reference in its entirety.

### BACKGROUND OF THE INVENTION

The present invention relates generally to devices for foot measurement and in particular to pressure sensitive measurement devices for selecting podiatric orthotics. The present invention also relates to orthotic devices having multiple configurations.

A wide variety of orthotic devices are available for consumers today. Orthotic devices are typically externally applied appliances used to modify the structural and functional characteristics of the neuromuscular and skeletal system. Many of these devices are used to straighten spines and to correct podiatric conditions affecting the foot, ankle, and structures of the leg.

It has been estimated that approximately 57 percent of United States consumers between the ages of 18 and 65 have a podiatric condition that may be treated by the use of orthotic devices. However, orthotic devices for podiatric conditions are typically either too expensive or marginally effective. More specifically, custom fit orthotics typically require custom fitting at a doctor and are not cost effective for most consumers. On the other end of the spectrum are over the counter orthotics that are typically not properly fit for the individual consumer and address only consumer comfort, but not the underlying condition causing pain to the consumer.

To determine the appropriate orthotic device required to address the underlying podiatric condition of the consumer, the unique pressure distribution and length of the consumer's feet may be important measurements. However, these measurements typically require highly specialized equipment, precluding their use for the proper selection of over the counter orthotic devices. In addition, stores that sell over the counter orthotics typically have little floor space and poorly trained staff, accordingly the measurement device also needs to be small and fully operable by the consumer.

### BRIEF DESCRIPTION OF THE DRAWINGS

FIG. 1 is a diagram that illustrates an example of a pressure plate device in accordance with an embodiment of the invention.

FIG. 2 is a diagram that illustrates an example of a pressure distribution map of a consumer's feet in accordance with an embodiment of the invention.

FIG. 3 is a diagram that illustrates an example of an optical foot length measurement system in accordance with an embodiment of the invention.

FIG. 4 is a diagram that illustrates examples of different arch heights.

FIG. 5 is a diagram that illustrates various contact pressure distributions of a consumer's foot in accordance with an embodiment of the invention.

FIG. 6A is a diagram that illustrates how a pressure contour may be divided into sections in accordance with an embodiment of the invention.

FIG. 6B is a diagram that illustrates how an array of pressure sensors may be used to map the pressure contour of a foot in accordance with an embodiment of the invention.

FIGS. 7-9 are diagrams that illustrate various kiosks in accordance with embodiments of the invention.

FIG. 10 is a diagram that illustrates a flow chart of one or more display screens in accordance with an embodiment of the invention.

FIG. 11 is a diagram that illustrates a computer system integrated in the pressure plate in accordance with an embodiment of the invention.

### SUMMARY

In one embodiment pressure contour data from a consumer's feet are analyzed by a computer within a pressure measurement device and a foot type that best matches the pressure contour data is selected. In some embodiments, the foot type may correspond to the type of arch that the consumer has. For example the foot type may be a low, a medium or a high arch. In other embodiments other foot types may be analyzed. Myriad algorithms may be employed to determine the best foot type match to the consumer and the appropriate orthotic device. Myriad orthotic devices may be selected including those described in pending U.S. Patent Application Pub. No. 2012/0304490 which is incorporated herein by reference in its entirety for all purposes.

In one embodiment the orthotic devices can be offered as a 3-in-1 version allowing three different uses of the product contained in one package. An orthotic shell and a top cover with a temporary adhesive may be packaged separately allowing the consumer to select between three different uses. A first use may include a memory foam cushion alone. A second use may include a supportive orthotic insole shell alone. A third use may include an application where the adhesive of the top cover is removed and the shell and the top cover are combined for support and comfort. Alternatively, an adhesive protective material is removed from the top cover and the shell and the top cover are combined.

In one embodiment pressure contour data derived from a consumer's feet may be analyzed by a computer within the pressure measurement device by dividing up the consumer's pressure contour into sections. In further embodiments the pressure contour is divided into three sections and the pressure contour within each section is analyzed. In some embodiments, the data in each section of the pressure contour is used to determine a representative width of that section. In other embodiments the length and/or the area of that section is used. In yet further embodiments, the data from each section may be used to calculate foot ratios that may be used to determine the foot type of the consumer. The data may be used in myriad ways to select the appropriate orthotic device for the consumer. In yet further embodiments, the pressure contour data may be used to detect other foot conditions such as the need for a metatarsal pad and/or added cushioning on the heel.

In one embodiment the foot length measurement is determined visually by the consumer looking at an graphical indication on the device. In other embodiments one or more sensors on the device may measure the foot length. In one embodiment a plurality of optical transmitters and receivers are disposed opposite and parallel one another to measure the length of both of the consumer's feet. In further embodiments the optical transmitters and receivers are operated in pairs.

In one embodiment the pressure plate device may be modular and integrated into a kiosk, or used on its own. In some embodiments the kiosk may have a lower panel and a vertical panel used to display products and/or information such as on a display. In other embodiments the kiosk may be compact and include a horizontal panel with products and or information such as on a display. In one embodiment the pressure plate may communicate with the kiosk through a wireless or a wired connection.

In one embodiment the pressure plate device may have a computer system integrated within it that includes one or more processors, data storage systems and communications systems. In some embodiments the computing system may interface with external inputs and outputs such as a kiosk display and/or a consumer's portable electronic device.

To better understand the nature and advantages of the present invention, reference should be made to the following description and the accompanying figures. It is to be understood, however, that each of the figures is provided for the purpose of illustration only and is not intended as a definition of the limits of the scope of the present invention.

### DETAILED DESCRIPTION OF THE INVENTION

### Orthotic Devices

Generally orthotic insoles may be made from gel or other materials which fill the empty space under the arches in order to provide some level of arch support which may be necessary to address a customer's discomfort with their feet. In some embodiments insoles may cover the entire surface area of the foot in order to provide cushioning under the ball of the foot. In further embodiments, insoles may take up significant space in a shoe and may require shoes to be a size larger than the customer's normal size would be in order accommodate the insole in the customer's shoe. In other embodiments, orthotic insoles are designed to fit into common dress shoes and may be designed in ¾ length, ending before the ball of the foot and therefore not taking up space in the forefoot region of the shoe. Under the arch and in the heel they may be designed with a combination of materials which do not just fill up empty space in order to provide support but they may be filled with a blend of different polymer mixtures which may provide support yet keeping the overall design thin. Such low profile designs may make these orthotic inserts easier to fit in every day shoes without requiring the user to change to larger size shoes.

In some embodiments, biomechanically engineered orthotic insoles may differ from conventional insoles as the purpose may be to stabilize movement in the joints, thereby addressing the biomechanics of the foot which is often the primary cause of foot discomfort. In further embodiments, insoles may primarily cushion certain foot areas to provide relief of discomfort and thereby only treat the symptoms. In other embodiments, biomechanically engineered orthotic insoles may address the root problem of foot discomfort and not just mitigate the effects. In order to do so these embodiments may be made from firmer materials, which if not fitted correctly may not be comfortable to wear. In order to address this issue, orthotic insoles in these embodiments may have to be made in different sizes which do not just vary based on shoe size but also take different foot forms (heel width, arch height, weight) into account in order to provide the relief consumers are looking for yet still be comfortable to wear.

In one embodiment, biomechanically engineered orthotic inserts may employ composite layer technology to offer support and comfort. The orthotic inserts may be designed to work in formal and casual shoes. In further embodiments, a ¾ length shape may improve the fit in the consumer's shoes. In other embodiments, additional features may be employed in orthotic inserts such as:
a) Abrasion resistant Ultra-Luxe suede
b) Memory Foam that instantly conforms to the consumer's foot without adding bulk
c) Tear resistant middle layer for added strength and integrity
d) Shock-absorbing plantar fascia pad to cushion heel
e) Heel punch-out to relieve pressure
f) High strength, ultra-thin carbon composite core that supports the consumer's arch and distributes pressure

Firmer materials may initially not be as comfortable to the touch as softer materials. In order to provide support and stability to the foot's biomechanics yet still feel comfortable to the touch, in some embodiments, the orthotic insoles may employ different layers of material as described in more detail below. It is understood that the layers described below may be used individually or in any combination with each other. In one embodiment the orthotic devices can be offered as a 3-in-1 version allowing three different uses of the product contained in one package. An orthotic shell and a top cover with a temporary adhesive may be packaged separately allowing the consumer to select between three different uses. A first use may include a memory foam cushion alone. A second use may include a supportive orthotic insole shell alone. A third use may include an application where the adhesive of the top cover is removed and the shell and the top cover are combined for support and comfort. Alternatively, an adhesive protective material is removed from the top cover and the shell and the top cover are combined. Once the top cover is worn out, a replacement cover may be bought and used with the same orthotic insole shell to exchange faster wearing out parts while keeping the durable orthotic insole shell. Other features may be employed in some embodiments of the orthotic devices as described below.
a) In some embodiments, the top layer may be an abrasion resistant suede warp knit fabric as a foot experiences considerable movement in a shoe and this layer may experience shear forces. In some embodiments, the material may have a score of 48,000 dry rubs on the Martindale Abrasion Resistance scale and 4,350 cycles on the Taber Abrasion Resistance Scale.
b) In some embodiments, a visco-elastic foam layer, commonly referred to as ‘memory foam’, may be employed. Such memory foams may be pressure-sensitive and may mold quickly to the shape of a body pressing against it, returning to its original shape once the pressure is removed but with a delay. In further embodiments, this foam may have an extremely low density of only 64-112 kg/m3, and as such, once compressed it may add little bulk to the insert.
c) In some embodiments, visco-elastic foams may not be tear-resistant so an additional textile layer may be added to ‘cover’ the visco-elastic foam on both sides. In further embodiments, the tear resistant middle layer may have a Ball Burst Strength of 45 pounds per square inch, according to ASTM D 3787-89. This may add tear resistance to the otherwise non-tear resistant visco-elastic foam.
d) In some embodiments, one of the primary uses of the orthotic device may be to relieve foot discomfort in the heel that may be caused by an inflammation of the plantar fascia. Further embodiments may employ supporting arches to prevent the customer's arches from collapsing and reducing the tear forces on the plantar fascia. In still further embodiments, a thin gel layer may be provided under the area where the plantar fascia connects to the heel bone to increase comfort. In further embodiments, the gel area may be extended to cover the area under the heel. During the heel strike phase in the gait cycle shockwaves may run from the heel all the way along the foot to knee, hips and the back often causing knee and back pain. Providing a layer of gel under that area may absorb the shock at heel strike and may greatly reduce the propagation of shockwaves to the upper leg and body.
e) In some embodiments the benefits of gel in the heel area may be accomplished by adding the gel on top of the firmer orthotic insole shell. However, this configuration may increase the bulk and may not result in a low profile design. In some circumstances, the heel pain mentioned above may be relieved by reducing the pressure at which the heel touches the surface. In some embodiments, cutting out the heel area in the insoles may lead to a pressure release under the heel. In further embodiments, filling the cut out area with gel may allow for the combined effect of shock absorption to address heel, knee and back pain but also additional relief of potential plantar fascia pain and all in a design which does not add bulk to the shoe.
f) In some embodiments, a high strength, ultra-thin carbon composite core may support the arch and distribute pressure.
g) In some embodiments, a unique blend of thermoplastic polymer with thermoplastic elastomer may benefit foot support in the low profile design, as explained above. In further embodiments, the deflection strength may vary between the different orthotic variants between 14 and 28 ft/lb. This may cause a more even weight distribution under the surface area of the foot, reducing peak pressure points.

### Foot Measurement and Orthotic Selection

Numerous consumers have podiatric conditions that may be improved or resolved by the use of an orthotic device. Such devices may be designed into a custom shoe while other devices may be designed to fit within a consumer's existing shoe. To be effective, the orthotic device is typically disposed between the bottom of the consumer's foot and the top of the insole of the shoe. This location may allow the orthotic device to correct, support and align the foot joints, relieving pain from the consumer. Because every consumer's foot has a unique pressure contour and length, a properly fit orthotic device may be based on the particular measurements of the consumer's foot.

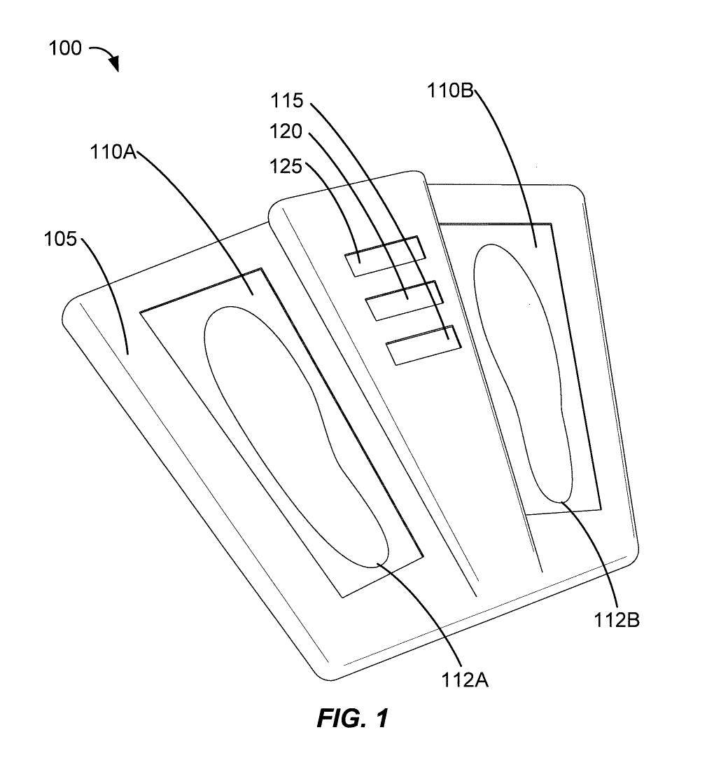

An embodiment of a device for measuring the pressure contours and the length of consumer's feet is illustrated in FIG. 1. Pressure plate 100 has an outer body 105 with two pressure measurement locations 110A, 110B for the consumer's left and right foot, respectively. Pressure plate 100 may also have one or more displays 115, 120, 125 that may be disposed between two pressure measurement locations 110A, 110B. In some embodiments, displays 115, 120, 125 may simply be colored lights, while in other embodiments one or more of them may be a graphical display such as a black and white or color liquid crystal display, or similar apparatus.

In some embodiments, pressure measurement locations 110A, 110B are oriented in a slight V-shape relative to one another with less separation towards the heel portion and more separation towards the forefoot portion. This may provide more comfort for the consumer during the measurement process because pressure measurement locations 110A, 110B may align better with the natural orientation of the consumer's feet. In addition, the V-shape design may provide more accurate foot measurements as the consumer's foot is in a more relaxed form than if they were substantially parallel which might change the shape of the foot due to the fact that the customer needs to extend or contract mussels in order to bring the foot into the parallel position. In further embodiments there may no V-shape between pressure measurement locations 110A, 110B and the pressure measurement locations may be substantially parallel. Such embodiments may provide for a more compact pressure plate 100, enabling it to fit into smaller retail locations.

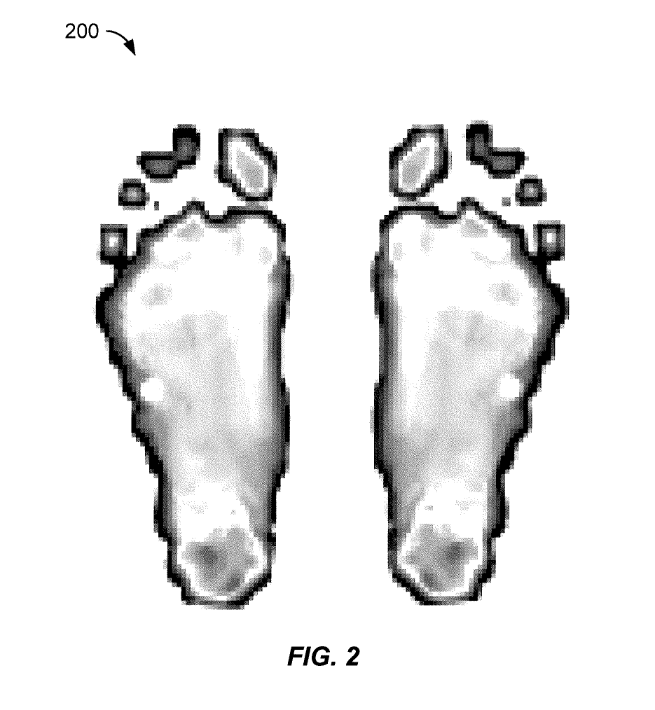

To operate pressure plate 100, a consumer may place their feet on pressure measurement locations 110A, 110B and within outlines 112A, 112B. A pressure sensitive contact array (discussed in more detail below) may be disposed on a top surface of outer body 105 and within pressure measurement locations 110A, 110B. The pressure sensitive contact array may include a plurality of pressure sensors that individually read the pressure placed on each of them by the consumer's foot. The sensors are read by electronic circuitry (not shown) and a pressure contour 200 of the consumer's feet may be produced, as illustrated in FIG. 2. Pressure contour 200 may be displayed on one or more displays 115, 120, 125 (see FIG. 1) of pressure plate 100, and/or the pressure contour may be virtual and the electronic circuitry may perform calculations on the data from the pressure sensors. Pressure contour 200 in FIG. 2 illustrates the amount of pressure exerted by certain portions of the consumer's feet on pressure measurement locations 110A, 110B. Myriad calculations may be performed with pressure contour 200 data, as discussed in more detail below.

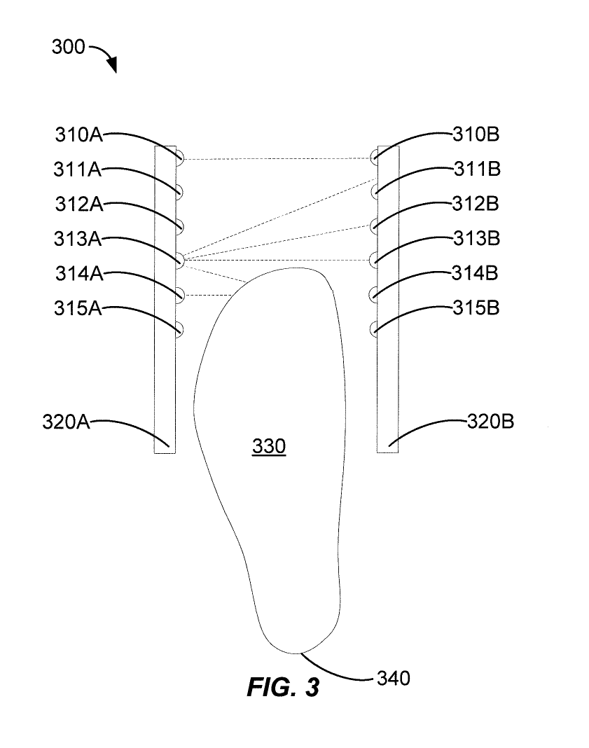

Pressure plate 100 may also be equipped with a foot length measurement system. In one embodiment the foot length measurement is simply performed by the consumer looking at a graphical measurement indicator inscribed on pressure measurement locations 110A, 110B (see FIG. 1). In further embodiments incremental lines or color coded zones may show the consumer which length of orthotic is appropriate for their use. In yet further embodiments, pressure contour 200 data may be used to determine the length of the consumer's feet. One issue with this method may be the accuracy of the measurement. More specifically, the consumer may have very low pressure on their large toe or heel which may indicate that their foot is shorter than it actually is. To mitigate these issues, some embodiments may employ an optical foot measurement system as illustrated in FIG. 3.

One or both pressure measurement locations 110A, 110B (see FIG. 1) may be equipped with an optical foot measurement system 300. In some embodiments, optical foot measurement system 300 may comprise two rails 320A, 320B that may be disposed adjacent and parallel one another such that a consumer's foot 320 may be placed between them. A number of sensors 310A-315B may be located on each rail. In further embodiments rails 320A, 320B may not exist and sensors 310A-315B may be integrated within outer housing 105 (see FIG. 1). In some embodiments, optical transmitters 310A-315A may be mounted opposite optical receivers 310B-315B. In further embodiments, sensors 310A-315B may operate in the infra-red spectrum.

As illustrated in FIG. 3, a consumer's foot 330 may be disposed on one or both of pressure measurement locations 110A, 110B (see FIG. 1) between rails 320A, 320B such that one or more of optical receivers 310B-315B may be blocked from receiving an optical signal from one or more optical transmitters 310A-315A. In some embodiments, optical transmitters 310A-315A may employ a narrow beam while in other embodiments they may employ a diffuse beam. Both types of beams will be discussed in more detail below.

In one embodiment, as illustrated in FIG. 3, optical transmitter 310A may transmit a narrow beam such that only optical receiver 310B directly across from it may detect the beam. That is, when transmitter 310A emits an optical beam and there is no foot 330 between rails 320A, 320B, only receiver 310B may detect the beam and sensors 311B-315B cannot detect the beam. In these embodiments all transmitters 310A-315A and all sensors 310B-315B may be operated simultaneously because there is no optical crosstalk between the transmitter/receiver pairs. In other embodiments the transmitter/receiver pairs may be operated sequentially. To determine foot 330 length the system may detect which optical receivers are receiving a beam and/or which optical receivers are not. Foot 330 length may be determined by having a known distance between heel 340 of the foot and the transmitter/receiver pairs, as discussed in more detail herein. In other embodiments optical transmitter/receiver pairs 310A-315B may be operated sequentially.

In further embodiments, optical transmitter 313A may emit a diffuse optical beam that may be received by a plurality of optical receivers including 311B, 312B, 313B and 314B. As an illustrative example, to determine foot 330 length, transmitter 312A may emit a diffuse beam its beam may be sensed by optical receivers 311B-313B, but not 314B. Thus foot length 330 may be determined by knowing the distance between heel 340 of foot 330 and the closest optical receiver that does not sense the diffuse beam. In some embodiments, a more accurate foot 330 length measurement may be obtained by sequentially activating each diffuse optical transmitter 310A-315A and when each transmitter is activated, reading all optical receivers 310B-315B. By analyzing which optical receivers are activated by which optical transmitters, the length of foot 330 may be determined more precisely than if the transmitter/receivers were simply operated in pairs. Other methods may be employed to determine foot 330 length such as using the optical sensor data in conjunction with foot pressure readings.

In some embodiments the foot length of the consumer may be displayed on one or more display screens 115, 120, 125 (see FIG. 1) on pressure plate 100. Some embodiments may contain up to six optical transmit/receive pairs, while further embodiments may contain up to 12 optical transmit/receive pairs while still further embodiments may contain 20 or more optical transmit/receive pairs. The more optical transmit/receive pairs employed, the higher the accuracy of the foot length measurement. Improved foot length measurement accuracy may be used to select half and/or quarter shoe sizes for the consumer.

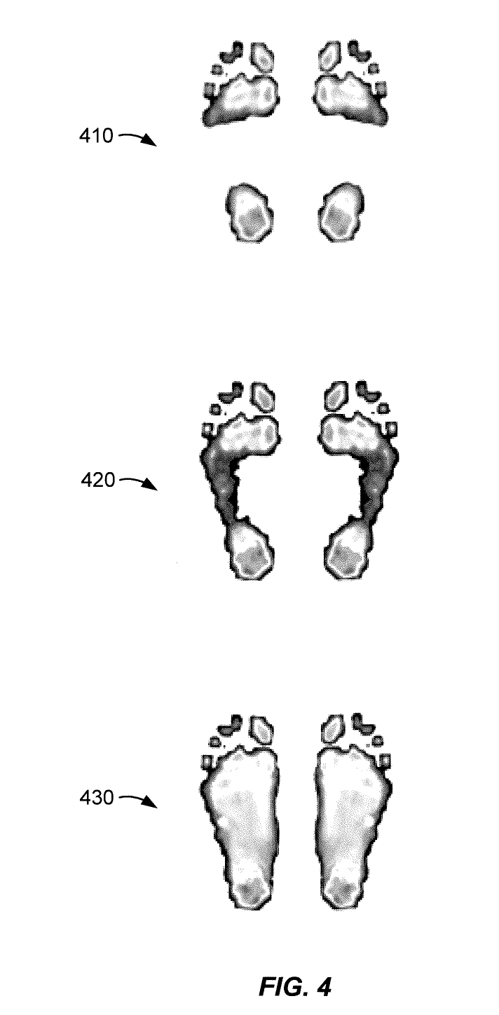

In some embodiments, pressure plate 100 (see FIG. 1) may be programmed to take pressure contour 200 (see FIG. 2) of the consumer's foot and perform one or more analyses of the data extracted from the plurality of pressure sensors. In one embodiment, an algorithm may be used to match pressure contour 200 to one of a plurality of foot types as illustrated in FIG. 4. For example, in some embodiments there may be three foot types called “High Arch” 410, “Medium Arch” 420 and “Low Arch” 430. In some embodiments there may be more than three foot types while in other embodiments there may be fewer than three foot types. In one embodiment there are five foot types. Myriad algorithms may be used to determine which foot type the consumer's pressure contour 200 most closely matches. In some embodiments a “best fit” algorithm may be used that compares the geometry of the consumer's foot with the three foot types 410, 420, 430 and determines which foot type best matches the consumer's pressure contour 200. In further embodiments this analysis may be determined by a percent match algorithm that may also include scaling of the foot types to approximately match the length of the consumer's foot.

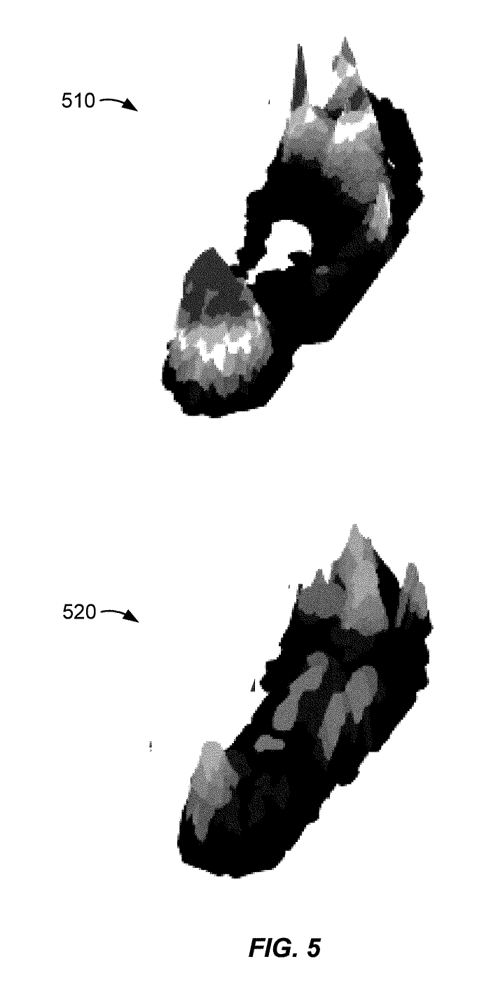

In further embodiments the goal of the algorithms may be to reduce the maximum pressure regions on the consumer's foot, making the pressure distribution on the consumer's foot more uniform. As an example, FIG. 5 illustrates a pressure contour of a consumer's foot with no orthotic device 510. The high peaks denote high pressure regions that likely cause discomfort to the consumer. Illustration 520 depicts the same foot with support by an orthotic device. As shown, the high pressure peaks in illustration 510 have been decreased, resulting in greater consumer comfort. The length of the orthotic may be selected based on the foot length measurements discussed above. Information such as the foot type, the pressure contour map, the foot length, the orthotic product number and other relevant information may be displayed on one or more of displays 115, 120, 125 (see FIG. 1). In one embodiment, the best fit orthotic is selected based on differentiation in arch height and foot length only.

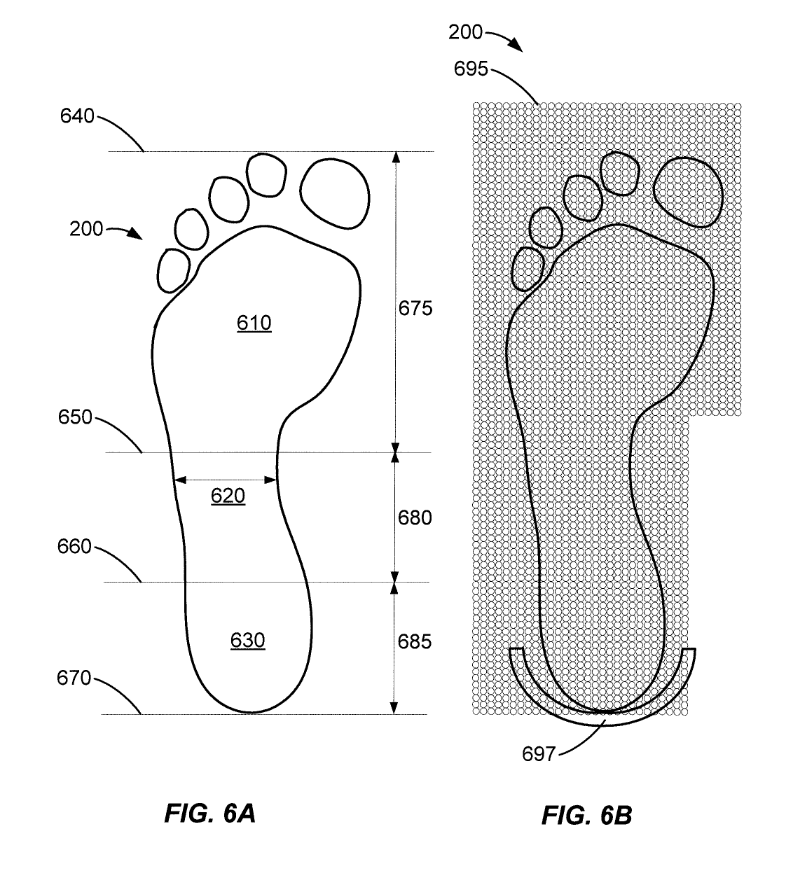

In one embodiment, illustrated in FIG. 6A, pressure contour 200 (see FIG. 2) may be divided up into three sections by lines 640, 650, 660 and 670. In other embodiments fewer sections may be used while in further embodiments more sections may be used. Forefoot section 610 is defined by lines 640 and 650 which represent forefoot length percentage 675 of the foot. Forefoot length percentage may be any percentage of the total foot length, in this example it is the front 45 percent of the foot length. Arch section 620 is defined by lines 650 and 660 which represent arch length percentage 680. Arch length percentage may be any percentage of the total foot length, in this example it is the middle 28 percent of the foot length. Heel section 630 is defined by lines 670 and 660 which represent heel length percentage 685. Heel length percentage may be any percentage of the total foot length, in this example it is the rear 27 percent of the foot length. The percentages provided in this example are for illustration only and other percentages may be used. For example forefoot length percentage 675 may range between 40-50 percent, 35 to 55 percent or 30 to 60 percent. Arch section 680 may range from 23 to 33 percent, 18 to 38 percent or 13 to 42 percent. Heel length percentage 685 may range from 22 to 32 percent, 18 to 37 percent or 13 to 42 percent.

FIG. 6B illustrates an embodiment where a foot is placed on an array of pressure sensors 695. In some embodiments pressure sensors 695 with variable output are used in pressure measurement locations 110A, 110B (see FIG. 1) while in other embodiments pressure sensors that only respond to a threshold value of pressure are used. In embodiments that only use a threshold value, sensors 695 may only sense a pressure above a certain threshold, thus providing a binary feedback. More specifically, threshold value pressure sensors may essentially act as an on/off switch where if the pressure exerted on them exceeds the threshold pressure they may be in the on state and if the pressure exerted is below the threshold pressure they may be in the off state. In embodiments that employ variable pressure sensors, the absolute value of the pressure reading as well as the relative value of the pressure sensors may be ascertained and used. Various calibration routines may be employed to improve the absolute accuracy of pressure sensors 695. Pressure sensors 695 in FIG. 6B are for illustrative purposes only and the quantity, arrangement and configuration of the sensors may be different in other embodiments.

In one embodiment pressure sensors 695 may include a conductive film in which the electrical conductivity of the film depends on the pressure exerted on the film. Under the film may be an array of separated contacts and depending on the pressure exerted on the film, the conductivity between the separated contacts changes which in turn can be correlated to the respective pressure exerted at the point of the separated contacts. Each separated contact may act similarly, providing data in the form of a pressure contour of a consumer's foot. In some embodiments the time that the pressure measurement takes may be variable. In further embodiments the time of the pressure measurement may be increased beyond the time required to acquire data, for example, if it is desired to have a consumer remain on the pressure plate to read marketing literature.

Pressure sensors 695 may be used to determine the width of forefoot section 610, arch section 620 and heel section 630. When employing variable pressure sensors 695, myriad algorithms may be used to determine the width of the front section 610, the arch section 620 and the heel section 630 (see FIG. 6) of foot 330.

For example, in one embodiment an absolute value can be set for each variable pressure sensor 695, forcing it to perform as a simple on/off sensor with binary feedback. In some embodiments the threshold value may be variable, allowing for the accommodation of different conditions such as consumer weight. However, in other embodiments, the relative value of pressure sensors 695 may be taken into account. For example, two customers may have the same footprint in terms of contact area on the pressure measurement locations 110A, 110B (see FIG. 1), however their arch height and foot type may be markedly different. The first person may have a medium arch, where arch section 620 has very low pressure readings and the second person may have a low arch where the arch section has very high pressure readings. By evaluating the absolute value of the pressure readings the arch height of the consumer may be more accurately determined. More specifically, lower pressure readings on the arch of the first person with the medium arch indicate that although the consumer's arch is touching pressure sensors 695 their arch is applying very little pressure, indicating their arch is not completely fallen and it is thus a medium arch as compared to a low arch. Conversely, the second person with the low arch may have high pressure readings in the arch indicating that the arch has fallen and is truly a low arch.

Further, by analyzing the pressure in the arch area as compared to the pressure on the front or heel area it can be determined if the consumer's arch contact pressure is low primarily because they are a light person and all the pressure readings are low. Thus, in this case the consumer may have a low arch, but it is only their weight that makes it appear that their arch may be medium. The variable pressure data from the pressure sensors may be used in other ways to determine arch height without departing from the invention. In some embodiments the length of sections 610, 620 and 630 may be determined while in other embodiments the area of the sections may be determined.

In one embodiment, the pressure contour for each section 610, 620, 630 may be resolved to a singular width number for each respective section. More specifically, the plurality of pressure data readings in each section are resolved by an algorithm into a unitary width value that represents the width for that particular section of the foot. Other methods of data collection and analyses may also be employed without departing from the invention. The unitary width numbers for each section may then be used to determine one or more ratios that define the consumer's foot type.

In one embodiment the ratios are used to determine a foot type and a related arch height. For example, a ratio of the width of arch section 620 to the width of forefoot section 610 may be determined. In another embodiment the ratio of the width of arch section 620 to the forefoot section 610 added to the heel section 630 is determined. These ratios can be used to subsequently ascertain the corresponding foot type 410, 420, 430 (see FIG. 4), using a simple lookup table or other method.

For example, in one embodiment if one or more of the ratios are below 0.3 then the consumer is determined to have a high arch. In another embodiment, if one or more of the ratios are between 0.3 and 0.6 the consumer is determined to have a medium arch height. In a further embodiment, if one or more of the ratios are greater than 0.6 then the consumer is determined to have a low arch type. Myriad ranges may be used to determine more than three different foot types. In another embodiment the thresholds for the ratios are less than 0.3, between 0.3 and 0.5, between 0.5 and 0.7 and above 0.7. Other ranges may be used without departing from the invention.

In further embodiments, pressure contour 200 (see FIG. 2) for each section 610, 620, 630 is resolved to a singular area number for each respective section. More specifically, the plurality of pressure data readings in each section are resolved by an algorithm into a unitary area value that represents the area for that particular section of the foot. The area numbers for each section may then be used to determine one or more ratios that define the consumer's foot type, as discussed above.

The pressure data from sensors 695 can be used to determine orthotic features other than arch height that may be beneficial to consumers. For example, some orthotics may have a “metatarsal pad” which is a small cushion in the forefoot area and helps to offload peak pressure points in the ball of the foot. If a consumer exhibits high pressures in this area, insoles may have an option for the metatarsal pad. In further embodiments, if a person shows higher pressure in the heel, an orthotic with more heel cushioning may be selected. In order not to have a high number of products, a modular insole system may be used where the different components are stocked on the kiosk and pressure plate 100 (see FIG. 1) tells the customer which components to select in order to make up the device that addresses their specific problems.

In further embodiments, pressure sensors 695 may be used to determine if foot 200 is properly positioned against heel cup 697 so that an accurate foot length measurement may be determined. That is, data from one or more pressure sensors 695 proximate heel cup may be used to make sure there is no gap between foot 200 and heel cup 697. In some embodiments, if it is determined that a gap exists, the system may notify the consumer to slide their heel firmly against heel cup 697 before continuing. Foot length may be accurately determined by setting a known distance between heel cup 697 and optical transmitter/receiver pairs 310A-315B (see FIG. 1).

In other embodiments pressure sensors 695 may also be used to determine if a portion of the consumer's foot is off of the sensing area or is incorrectly positioned. In one embodiment, pressure data from the consumer may be compared to one or more stored configurations to determine if the pressure data fits within acceptable parameters within the stored configurations. For example, if a consumer's foot is too far to one side the foot shape may be extracted from the pressure readings and compared to one or more configurations to determine that their foot is incorrectly placed. The consumer may then be prompted to adjust the position of their feet on the device accordingly.

In further embodiments, multiple sequential foot pressure analyses may be performed and compared to improve the accuracy of the analysis. In one embodiment, up to 10 sequential analyses may be performed. In another embodiment up to 20 sequential analyses may be performed. In a further embodiment 30 or more sequential analyses may be performed. In some embodiments the sequential analyses may be useful to average out the changing pressure readings due to the consumer changing the distribution of their weight on their feet during the analysis. In further embodiments, data from the pressure readings may be compared to stored preprogramed configurations to determine the most appropriate orthotic. In other embodiments, historical data may be collected from prior recommendations and used to make future recommendations. In one embodiment, the system may only display a result if in more than 80 percent of the individual foot evaluations the same product was selected by the system.

In some embodiments, pressure plate 100 (see FIG. 1) may have a manual power switch while in other embodiments the pressure plate may have an automatic power switch. A manual power switch may be operable by the consumer and may be any mechanical or solid-state switch that can activate electronic circuitry inside the pressure plate. An automatic power switch may sense the consumer and automatically activate the electronic circuitry inside the pressure plate. More specifically, an automatic switch may be a proximity, an optical or a pressure sensing device that the consumer does not have to specifically toggle to activate the electronic circuitry. In some embodiments the power switch may be one or more pressure sensors. In one embodiment, when pressure is sensed on one of pressure measurement locations 110A, 110B (see FIG. 1) the unit may power on.

In some embodiments when pressure plate 100 (see FIG. 1) is on it may “power down” to a low power consumption mode in which the only power used is for an internal computing system to ping the pressure sensors repeatedly to determine if they are detecting a pressure reading. If there is no pressure sensed, the system may remain in the low power standby mode. If the sensors signal back to the computing system that they detect pressure, the computing system may determine that someone is standing on it and it may power up the system providing power to the foot length measurement system 300 (see FIG. 3), the displays 115, 120, 125 (see FIG. 1) and all other systems required to take the pressure reading. Pressure plate 100 (see FIG. 1) may then perform foot pressure and foot length measurements. Pressure plate 100 may display the results for a number of seconds before it moves back into the low power consumption standby mode, automatically. In some embodiments pressure plate 100 (see FIG. 1) may operate off internal batteries, in other embodiments it may operate off an external alternating current source and in further embodiments it may operate off both where the internal batteries may be rechargeable and/or used for backup.

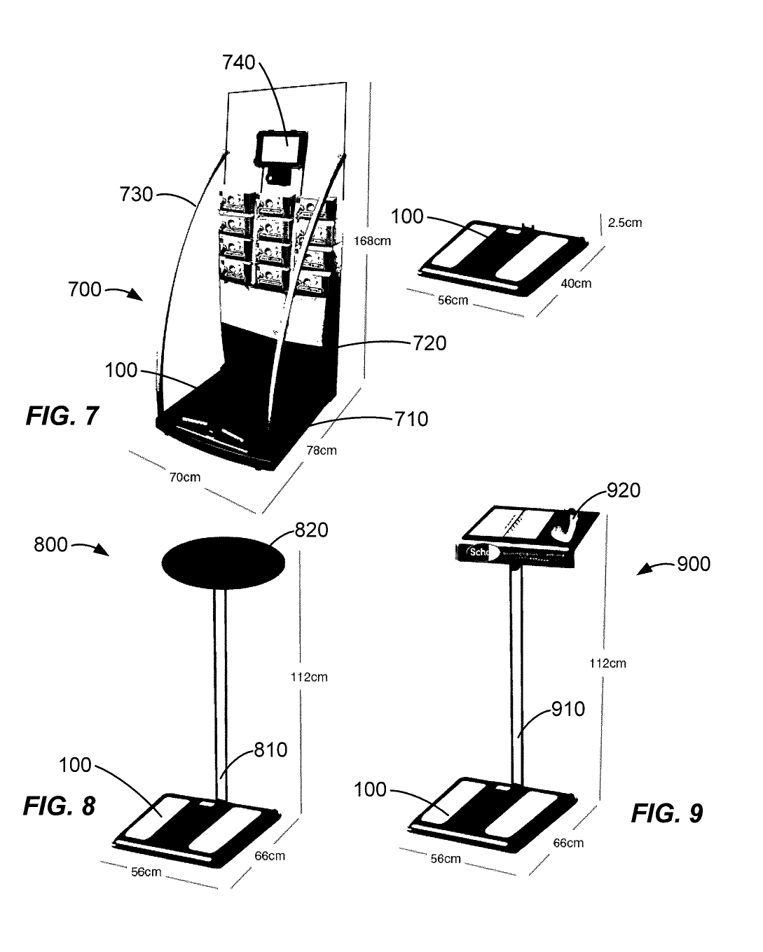

In some embodiments, pressure plate 100 (see FIG. 1) may be integrated into a kiosk 700, 800, 900 as illustrated in FIGS. 7-9. Pressure plate 100 may be modular and easily attached and detached from kiosks 700, 800, 900, or it may be permanently integrated or used separately. Kiosks 700, 800, 900 may be equipped with one or more display screens, manuals, literature and/or merchandise such as orthotic inserts. Pressure plate 100 (see FIG. 1) may electrically connect to kiosk 700, 800, 900 via a wired connection or a wireless connection, as described in more detail below. In one embodiment a data connector and a power connector interface kiosk 700, 800, 900 to pressure plate 100. In other embodiments a wireless data connection may be formed between kiosk 700, 800, 900 and pressure plate 100 (see FIG. 1), where the pressure plate is powered by an internal battery or external power source. In further embodiments the pressure plate display may be automatically turned off and/or connected to the one or more displays on the kiosk when the pressure plate is connected to, or brought within wireless proximity of the kiosk.

In FIG. 7 a full size kiosk 700 is illustrated. The dimensions are for illustration only and other dimensions and kiosk designs may be used with pressure plate 100. Kiosk 700 receives pressure plate 100 (see FIG. 1) as shown and has a lower panel 710 and a vertical panel 720. One or more arcuate rails 730 may connect lower panel 710 to vertical panel 720. Vertical panel 720 may contain merchandise including orthotic inserts, instructions, brochures and one or more displays 740. Display 740 may display marketing information, foot measurement information as discussed above, orthotic selection information and/or any other information relevant to the orthotic device.

In FIG. 8 a mini display kiosk 800 is illustrated. The dimensions are for illustration only and other dimensions and kiosk designs may be used with pressure plate 100. Kiosk 800 receives pressure plate 100 (see FIG. 1) and has a vertical support bar 810 supporting a horizontal panel 820. Horizontal panel 820 may contain merchandise including orthotic inserts, instructions, brochures and one or more displays (not shown). The displays may display marketing information, foot measurement information as discussed above, orthotic selection information and/or any other information relevant to the orthotic device.

In FIG. 9 a mini display kiosk 900 is illustrated. The dimensions are for illustration only and other dimensions and kiosk designs may be used with pressure plate 100. Kiosk 900 receives pressure plate 100 (see FIG. 1) and has a vertical support bar 910 supporting a horizontal panel 920. Horizontal panel 920 may contain merchandise including orthotic inserts, instructions, brochures and one or more displays (not shown). The displays may display marketing information, foot measurement information as discussed above, orthotic selection information and/or any other information relevant to the orthotic device.

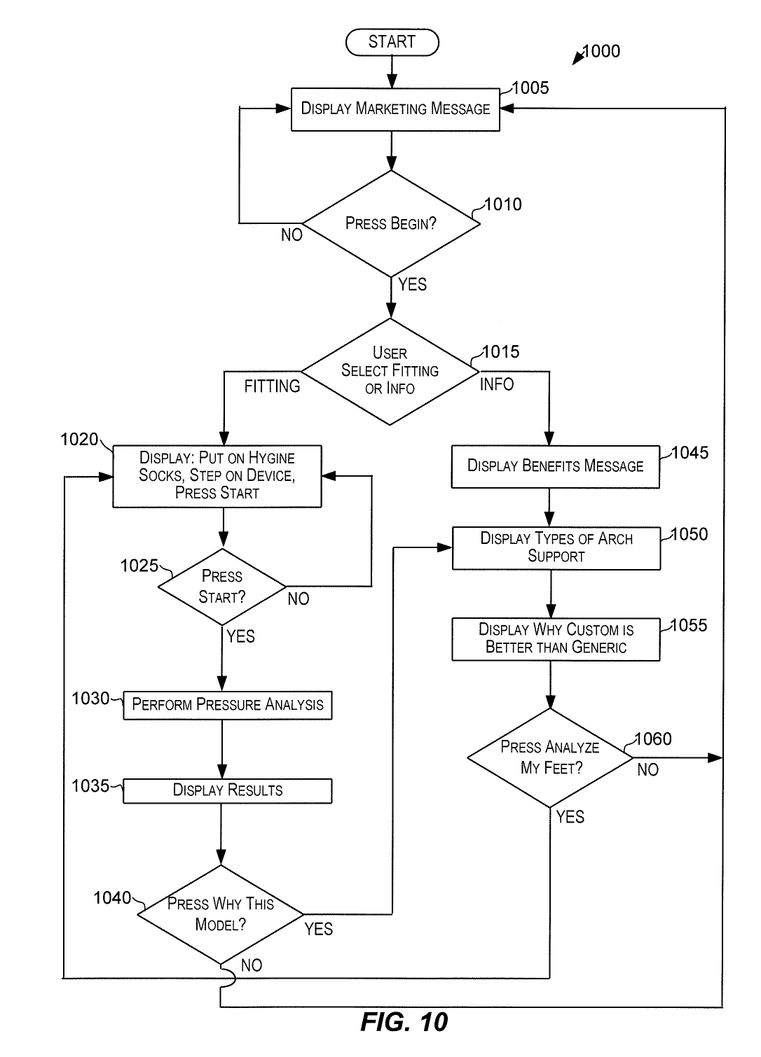

FIG. 10 depicts a simplified flowchart 1000 illustrating the general operation of a pressure plate 100 according to some embodiments. The processing depicted in FIG. 10 may be implemented in software (e.g., code, instructions, program) executed by one or more processors, in hardware, or combinations thereof. The software may be stored on a non-transitory computer-readable storage medium (e.g., stored on a memory device). The particular series of processing steps depicted in FIG. 10 is not intended to be limiting.

As depicted in FIG. 10, the method may be initiated at 1005 where one or more displays on the pressure plate or on the kiosks may display one or more marketing messages. At 1010 the consumer may be prompted to press a button labeled “Begin” on the display, step on the pressure plate or otherwise let the system know interaction is desired. At 1015 the consumer may be prompted to select between a fitting process or an information process to learn more about the product. If the consumer selects the fitting process, the program will advance to 1020. At 1020 the system may display one or more messages to the consumer to put on hygiene socks and step on the device. Other messages may also be displayed.

At 1025 the consumer may be requested to press start when ready. If the consumer presses start the program may transition to 1030, otherwise it may redisplay the message in 1020. At 1030 the pressure plate device may perform a pressure analysis of the consumer's feet. The display may inform the consumer that their feet are being analyzed and to not move. In one embodiment an array of pressure sensors detect the pressure of the consumer's feet on the pressure plate. After the pressure measurements are completed at 1035 the display may show an image of the pressure map of the consumer's feet and inform them that they have a high, medium or low arch height. The display may also indicate the particular model of the orthotic device the consumer should select. In other embodiments, the system may display a live 2-D or 3-D image of the pressure readings. At 1040 the consumer may be prompted to learn more about the particular orthotic device that the system selected for them. If the consumer selects this option the system will advance to 1050 and display the types of arch support along with other relevant information. If the consumer does not select this option the display may transition back to 1005 and display marketing messages, after a predetermined time.

At 1015, if the consumer selects the option to receive additional information and learn more about the product, the program may advance to 1045 and 1050 where the display shows the consumer the benefits of the product, different types of arch support and other relevant information. At 1055 further messages may be displayed such as why a custom orthotic is better than a generic orthotic. The consumer may also be prompted at 1060 to analyze their feet. If the consumer selects this option the program may advance to 1020 and initiate the analysis routine. If the consumer does not select this option within a given time period the display may advance to 1005 where one or more marketing messages are displayed.

One of skill in the art will recognize that simplified flow chart 1000 is one of many ways to operate the system and that myriad other flow charts may be employed without departing from the invention. The various marketing messages and prompts are for illustration only and other messages and prompts may be used in any order. In further embodiments the system may maintain statistical information on the number of measurements performed, how many were aborted, what the results were and which products were recommended.

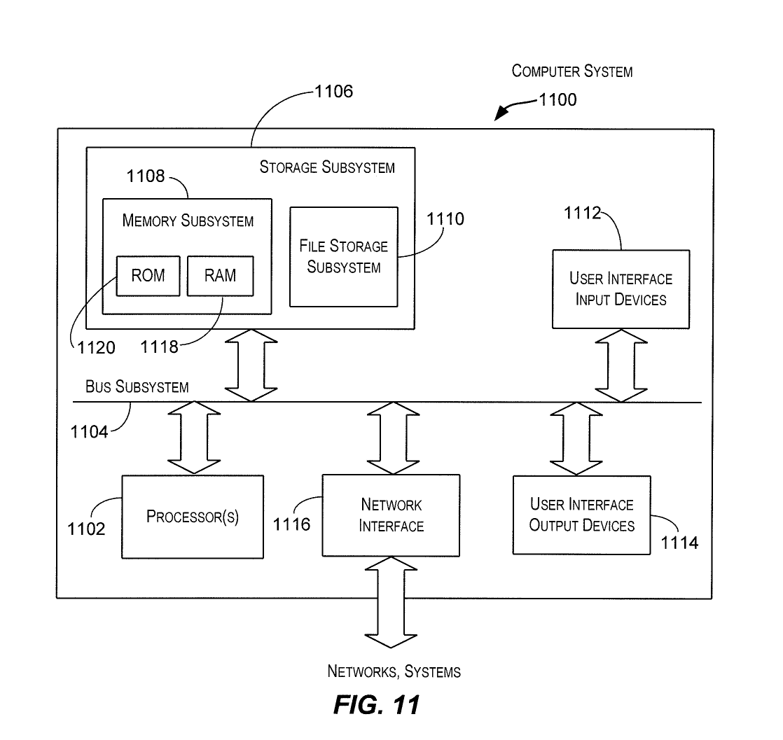

Pressure plate 100 (see FIG. 1) may incorporate various electrical systems and/or computing devices. FIG. 11 is a simplified block diagram of a computer system 1100 that may be incorporated in pressure plate 100 according to some embodiments. As shown in FIG. 11, computer system 1100 includes one or more processors 1102 that communicates with a number of peripheral subsystems via a bus subsystem 1104. These peripheral subsystems may include a storage subsystem 1106, including a memory subsystem 1108 and a file storage subsystem 1110, user interface input devices 1112, user interface output devices 1114, and a network interface subsystem 1116.

Bus subsystem 1104 provides a mechanism for letting the various components and subsystems of computer system 1100 communicate with each other as intended. Although bus subsystem 1104 is shown schematically as a single bus, alternative embodiments of the bus subsystem may utilize multiple busses.

One or more processors 1102, which can be implemented as one or more integrated circuits (e.g., a conventional microprocessor or microcontroller), can control the operation of computer system 1100. In various embodiments, one or more processors 1102 can execute a variety of programs in response to program code and can maintain multiple concurrently executing programs or processes. At any given time, some or all of the program code to be executed can be resident in one or more processors 1102 and/or in storage subsystem 1106. Through suitable programming, one or more processors 1102 can provide various functionalities described above for performing analyses of pressure sensor data.

Network interface subsystem 1116 provides an interface to other computer systems and networks. Network interface subsystem 1116 serves as an interface for receiving data from and transmitting data to other systems from computer system 1100. For example, network interface subsystem 1116 may enable computer system 1100 to connect to a client device via the Internet. In some embodiments network interface 1116 can include radio frequency (RF) transceiver components for accessing wireless voice and/or data networks (e.g., using cellular telephone technology, advanced data network technology such as 3G, 4G or EDGE, Bluetooth, WiFi (IEEE 802.11 family standards, or other mobile communication technologies, or any combination thereof), GPS receiver components, and/or other components. In some embodiments network interface 1116 can provide wired network connectivity (e.g., Ethernet) in addition to or instead of a wireless interface.

User interface input devices 1112 may include pressure sensor input, foot length measurement input, optical sensor input, keyboard, pointing devices such as a mouse or trackball, a touchpad or touch screen incorporated into a display, a scroll wheel, a click wheel, a dial, a button, a switch, a keypad, audio input devices such as voice recognition systems, microphones, and other types of input devices. In general, use of the term “input device” is intended to include all possible types of devices and mechanisms for inputting information to computer system 1100. In some embodiments a user may be able to communicate with computing system 1100 with their mobile device. In some embodiments foot measurement and/or product data may be directly transferred to the consumer's mobile device. In other embodiments data may be transferred from the consumer's mobile device to computing system 1100.

User interface output devices 1114 may include a display subsystem, indicator lights, or non-visual displays such as audio output devices, etc. The display subsystem may be a cathode ray tube (CRT), a flat-panel device such as a liquid crystal display (LCD), a projection device, a touch screen, and the like. In general, use of the term “output device” is intended to include all possible types of devices and mechanisms for outputting information from computer system 1100.

Storage subsystem 1106 provides a computer-readable storage medium for storing the basic programming and data constructs that provide the functionality of some embodiments. Storage subsystem 1106 can be implemented, e.g., using disk, flash memory, or any other storage media in any combination, and can include volatile and/or non-volatile storage as desired. Software (programs, code modules, instructions) that when executed by a processor provide the functionality described above may be stored in storage subsystem 1106. These software modules or instructions may be executed by processor(s) 1102. Storage subsystem 1106 may also provide a repository for storing data used in accordance with the present invention. Storage subsystem 1106 may include memory subsystem 1108 and file/disk storage subsystem 1110.

Memory subsystem 1108 may include a number of memories including a main random access memory (RAM) 1118 for storage of instructions and data during program execution and a read only memory (ROM) 1120 in which fixed instructions are stored. File storage subsystem 1110 provides persistent (non-volatile) storage for program and data files, and may include a hard disk drive, a floppy disk drive along with associated removable media, a Compact Disk Read Only Memory (CD-ROM) drive, an optical drive, removable media cartridges, and other like storage media.

Computer system 1100 can be of various types including a personal computer, a portable device (e.g., an iPhone®, an iPad®), a workstation, a network computer, a mainframe, a kiosk, a server, an embedded microprocessor based system or any other data processing system. Due to the ever-changing nature of computers and networks, the description of computer system 1100 depicted in FIG. 11 is intended only as a specific example. Many other configurations having more or fewer components than the system depicted in FIG. 11 are possible.

Various embodiments described above can be realized using any combination of dedicated components and/or programmable processors and/or other programmable devices. The various embodiments may be implemented only in hardware, or only in software, or using combinations thereof. The various processes described herein can be implemented on the same processor or different processors in any combination. Accordingly, where components are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for interprocess communication, and different pairs of processes may use different techniques, or the same pair of processes may use different techniques at different times. Further, while the embodiments described above may make reference to specific hardware and software components, those skilled in the art will appreciate that different combinations of hardware and/or software components may also be used and that particular operations described as being implemented in hardware might also be implemented in software or vice versa.

The various embodiments are not restricted to operation within certain specific data processing environments, but are free to operate within a plurality of data processing environments. Additionally, although embodiments have been described using a particular series of transactions, this is not intended to be limiting.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

What is claimed is:

1. A foot measurement system comprising:
a body;
two pressure measurement locations located on a top surface of the body with a plurality of pressure sensors disposed within the two pressure measurement locations; and
a foot length measurement system disposed on the body comprising a row of optical transmitters arranged parallel to and opposite a row of optical receivers.

2. The foot measurement system of claim 1 wherein the two pressure measurement locations are oriented in a slight V-shape relative to one another.

3. The foot measurement system of claim 1 wherein the optical transmitters emit a narrow beam.

4. The foot measurement system of claim 1 wherein the optical transmitters emit a diffuse beam.

5. The foot measurement system of claim 1 wherein the optical transmitters simultaneously emit optical beams.

6. The foot measurement system of claim 1 wherein a processor receives data from the plurality of pressure sensors and a processor divides the data into three sections representing a forefoot section, an arch section and a heel section.

7. The foot measurement system of claim 6 wherein the processor determines a singular width value for each the three sections representing a forefoot width, an arch width and a heel width.

8. The foot measurement system of claim 7 wherein the processor repeats the processes of receiving data from the plurality of pressure sensors and dividing the pressure sensor readings into three sections.

9. The foot measurement system of claim 7 wherein the processor uses a ratio of the arch width to the forefoot width to determine an arch height of the foot.

10. The foot measurement system of claim 7 wherein the processor uses a ratio of the arch width to the forefoot width added to the heel width to determine an arch height of the foot.

11. A foot measurement system comprising:
a body integrated into a kiosk;
two pressure measurement locations located on a top surface of the body with a plurality of pressure sensors disposed within the two pressure measurement locations; and
a foot length measurement system disposed on the body comprising a row of optical transmitters arranged parallel to and opposite a row of optical receivers.

12. The foot measurement system of claim 11 wherein the optical transmitters simultaneously emit optical beams.

13. The foot measurement system of claim 11 wherein a processor receives data from the plurality of pressure sensors and the processor divides the pressure sensor readings into three sections representing a forefoot section, an arch section and a heel section.

14. The foot measurement system of claim 13 wherein the processor determines a singular width value for each the three sections representing a forefoot width, an arch width and a heel width.

15. The foot measurement system of claim 14 wherein the processor repeats the processes of receiving data from the plurality of pressure sensors and dividing the pressure sensor readings into three sections.

16. The foot measurement system of claim 14 wherein the processor uses a ratio of the arch width to the forefoot width to determine an arch height of the foot.

17. The foot measurement system of claim 14 wherein the processor uses a ratio of the arch width to the forefoot width added to the heel width to determine an arch height of the foot.

18. A method of measuring a human foot using a pressure plate, the method comprising:
reading a plurality of pressure sensors disposed on a top surface of the pressure plate;
reading a plurality of optical receivers disposed on the top surface of the pressure plate;
analyzing data from the plurality of the pressure sensors to determine a pressure contour for the human foot; and
analyzing data from the optical receivers to determine a length of the human foot.

19. The method of claim 18 further comprising activating a plurality of optical transmitters arranged opposite and parallel to the plurality of optical receivers.

20. The method of claim 18 further comprising a processor configured to select an orthotic device based on the pressure contour and the length.
